(19) United States
(12) Patent Application Publication
(10) Pub. No.: US 2019/0194348 A1
(43) Pub. Date: Jun. 27, 2019

(54) TREATMENT OF THERAPY-RESISTANT HER-2 POSITIVE BREAST CANCER

(71) Applicant: OREGON HEALTH & SCIENCE UNIVERSITY, Portland, OR (US)

(72) Inventor: Shiuh-Wen Luoh, Portland, OR (US)

(21) Appl. No.: 16/186,245

(22) Filed: Nov. 9, 2018

Related U.S. Application Data
(60) Provisional application No. 62/584,674, filed on Nov. 10, 2017.

(51) Int. Cl. C07K 16/32; A61K 31/517; A61K 47/68

(52) U.S. Cl. CPC C07K 16/32; A61K 31/517; C07K 2317/21; A61K 47/6803; C07K 2317/24; A61K 47/6855

(57) ABSTRACT

This invention concerns the treatment of HER-2 positive breast cancer cells by mediating the signal of GRB7 using an anti-HER-1 antibody, such as panitumumab.

### FIELD OF THE INVENTION

The present invention concerns the use of anti-HER-1 antibodies to target HER-1 signaling to suppress the growth of therapy-resistant HER-2 positive breast cancer cells.

### BACKGROUND OF THE INVENTION

The growth factor receptor-bound protein 7 (GRB7) gene encodes a multi-domain signal transduction molecule and is part of the core of the HER-2 amplicon. In breast cancer with HER-2 amplification and over-expression, GRB7 is commonly co-amplified and over-expressed. Structure and function relationship study of GRB7 suggests a functional role of GRB7 in breast cancer. The role of GRB7 in HER-2 positive human breast cancer cell lines that are resistant to HER-2 targeted therapy such as trastuzumab and lapatinib remains unexplored however. We speculate that GRB7 facilitates the growth of therapy resistant, HER-2 positive human breast cancer cells and targeting the signaling pathways activated by GRB7 may decrease cancer cell growth and therefore represent a novel therapeutic intervention.

### SUMMARY OF THE INVENTION

Provided herein is a method of treatment for therapy-resistant HER-2 positive breast cancer in a human, the method comprising administering to a human in need thereof a pharmaceutically effective amount of an anti-HER-1 antibody.

Also Provided herein is a method of treatment for therapy-resistant HER-2 positive breast cancer in a human, the method comprising administering to a human in need thereof a pharmaceutically effective amount of an EGFR inhibitor.

Also provided herein is a method of suppressing the growth of therapy-resistant HER-2 positive breast cancer in a human in need thereof, the method comprising administering to the human a pharmaceutically effective amount of an anti-HER-1 antibody.

Also provided herein is a method of suppressing the growth of therapy-resistant HER-2 positive breast cancer in a human in need thereof, the method comprising administering to the human a pharmaceutically effective amount of an EGFR inhibitor.

Also provided is a method of reducing GRB7 activity in a human experiencing a therapy-resistant HER-2 positive breast cancer in a human, the method comprising administering to a human in need thereof a pharmaceutically effective amount of an anti-HER-1 antibody.

Also provided is a method of reducing GRB7 activity in a human experiencing a therapy-resistant HER-2 positive breast cancer in a human, the method comprising administering to a human in need thereof a pharmaceutically effective amount of an EGFR inhibitor.

Further provided is a method of treatment for triple negative breast cancer (TNBC) with GRB7 in a human, the method comprising administering to a human in need thereof a pharmaceutically effective amount of an anti-HER-1 antibody.

Further provided is a method of treatment for triple negative breast cancer (TNBC) with GRB7 in a human, the method comprising administering to a human in need thereof a pharmaceutically effective amount of an EGFR inhibitor.

Also provided is a method of inhibiting GRB7 activity in a human experiencing triple negative breast cancer, the method comprising administering to a human in need thereof a pharmaceutically effective amount of an anti-HER-1 antibody.

Also provided is a method of inhibiting GRB7 activity in a human experiencing triple negative breast cancer, the method comprising administering to a human in need thereof a pharmaceutically effective amount of an EGFR inhibitor.

Provided is also a method of treating HER-2 positive human breast cancer that is resistant to trastuzumab, the method comprising administering to a human in need thereof a pharmaceutically effective amount of an anti-HER-1 antibody.

Provided is also a method of treating HER-2 positive human breast cancer that is resistant to trastuzumab, the method comprising administering to a human in need thereof a pharmaceutically effective amount of an EGFR inhibitor.

Provided is also a method of treating HER-2 positive human breast cancer that is resistant to at least one inhibitor of the HER receptor family, including those selected from the group of lapatinib, neratinib, and afatinib, the method comprising administering to a human in need thereof a pharmaceutically effective amount of an anti-HER-1 antibody.

Provided is also a method of treating HER-2 positive human breast cancer that is resistant to at least one inhibitor of the HER receptor family, including those selected from the group of lapatinib, neratinib, and afatinib, the method comprising administering to a human in need thereof a pharmaceutically effective amount of an EGFR inhibitor.

Provided is also a method of treating HER-2 positive human breast cancer that is resistant to both trastuzumab and at least one inhibitor of the HER receptor family, including those selected from the group of lapatinib, neratinib, and afatinib, the method comprising administering to a human in need thereof a pharmaceutically effective amount of an anti-HER-1 antibody.

Provided is also a method of treating HER-2 positive human breast cancer that is resistant to both trastuzumab and at least one inhibitor of the HER receptor family, including those selected from the group of lapatinib, neratinib, and afatinib, the method comprising administering to a human in need thereof a pharmaceutically effective amount of an EGFR inhibitor.

Also provided is a method of treating HER-2 positive breast cancer in a human, the method comprising administering to a human in need thereof:
a) a pharmaceutically effective amount of panitumumab;
b) a pharmaceutically effective amount of trastuzumab; and
c) a pharmaceutically effective amount of at least one inhibitor of the HER receptor family, including those selected from the group of lapatinib, neratinib, and afatinib, for a pharmaceutically acceptable salt thereof.

Also provided is a method of treating HER-2 positive breast cancer in a human, the method comprising administering to a human in need thereof:
d) a pharmaceutically effective amount of an EGFR inhibitor;
e) a pharmaceutically effective amount of trastuzumab; and
f) a pharmaceutically effective amount of at least one inhibitor of the HER receptor family, including those selected from the group of lapatinib, neratinib, and afatinib, for a pharmaceutically acceptable salt thereof.

### BRIEF DESCRIPTION OF THE FIGURES

FIG. 1 is a gel demonstrating transient knock down of BRB7 protein expression achieved with siRNA transfection, with cell lines transfected with non-targeting siRNAs as negative controls.

FIG. 2 is a bar graph demonstrating the siRNA-mediated knock down of GRB7 decreased in in vitro cell growth compared to controls in several cell lines as measured by the CellTiter-Glo® assay (Promega).

FIG. 3 is a Western Blot analysis confirming stable knock down breast cancer cell lines achieved with lentiviral vector mediated shRNA over-expression.

FIG. 4(a) depicts shRNA mediated GRB7 knockdown decreased cell proliferation in the HCC-1954 cell line relative to controls in trastuzumab and lapatinib resistant cell lines as measured by incucyte cell imaging studies (*p<0.05).

FIG. 4(b) depicts shRNA mediated GRB7 knockdown decreased cell proliferation in the 21MT1cell line relative to controls in trastuzumab and lapatinib resistant cell lines as measured by incucyte cell imaging studies (*p<0.05).

FIG. 4(c) depicts shRNA mediated GRB7 knockdown decreased cell proliferation in the JimT1 cell line relative to controls in trastuzumab and lapatinib resistant cell lines as measured by incucyte cell imaging studies (*p<0.05).

FIG. 5 provides line graphs showing that the knock down in three cell lines of GRB7 decreased growth of tumor xenografts formed by HER2 positive, thereapy-resistant cell lines in immune-deficient mice compared to controls and measured by volume and weight.

FIG. 6 provides gel images showing GRB7 knock down leads to less HER-1 tyrosine phosphorylation from cell lysates first immunoprecipitated with anti-HER-1 Ab, then blotted with anti-HER-1 and P-tyrosine antibody.

FIG. 7 provides a gel depicting HER-1 knock down in therapy-resistant, HER-2 positive BC cells via siRNA transient transfection.

FIG. 8 provides a bar graph of cell proliferation 4 days post-infection showing transient HER-1 knockdown in therapy-resistant HER-2 positive breast cancer cells reduces proliferation.

FIG. 9 provides a bar graph demonstrating reduction in proliferation of HER-2 positive, therapy-resistant cell lines following anti-HER-1 antibody treatment.

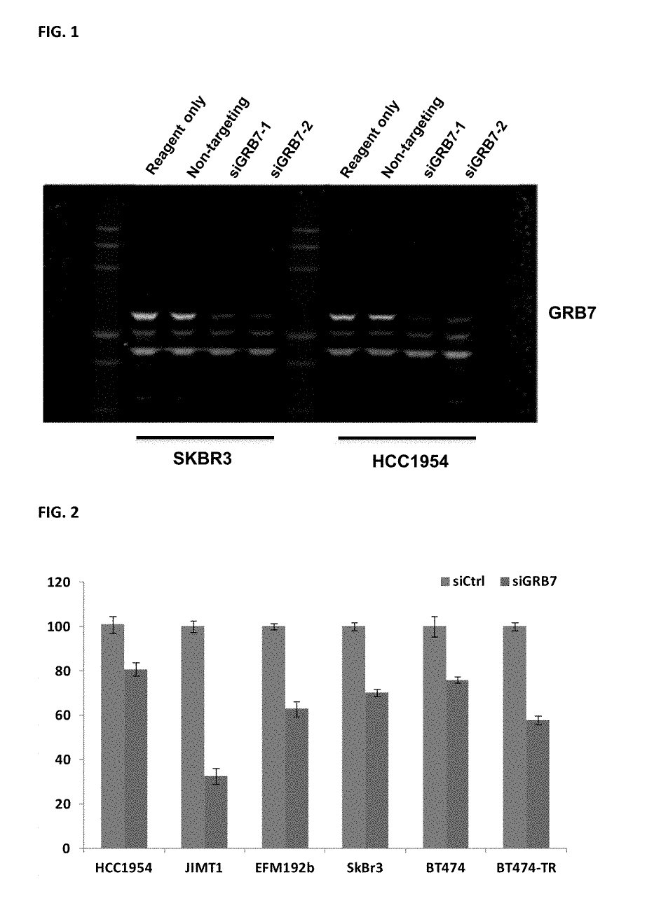

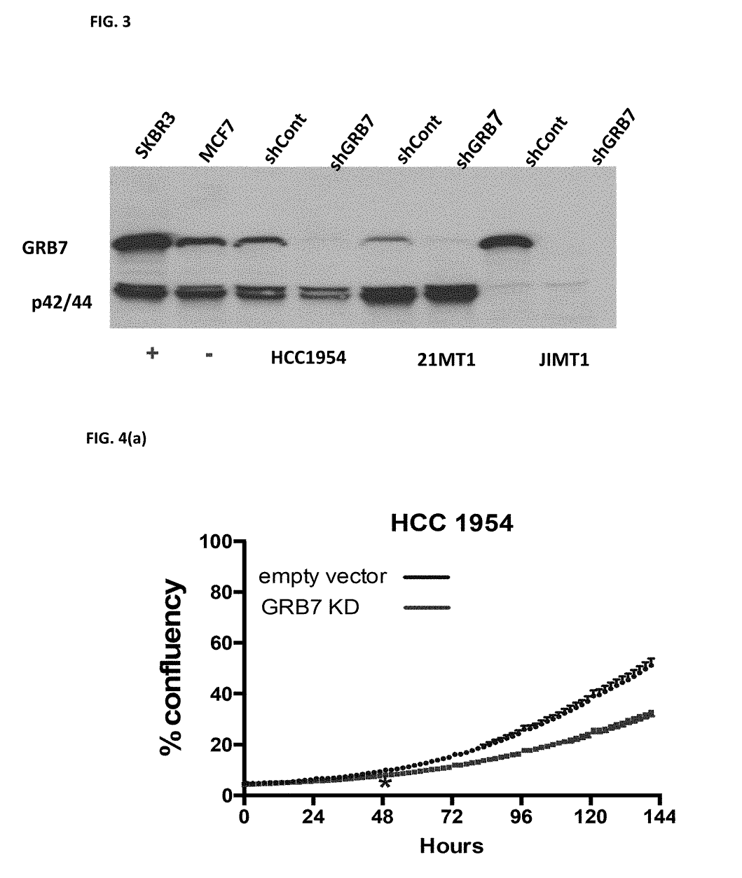

### DETAILED DESCRIPTION OF THE INVENTION

Also provided is a method of reducing GRB7 activity in a human experiencing a therapy-resistant HER-2 positive breast cancer in a human, the method comprising administering to a human in need thereof a pharmaceutically effective amount of an anti-HER-1 antibody.

Also provided is a method of reducing GRB7 activity in a human experiencing a therapy-resistant HER-2 positive breast cancer in a human, the method comprising administering to a human in need thereof a pharmaceutically effective amount of an EGFR inhibitor.

Further provided is a method of treatment for triple negative breast cancer (TNBC) with GRB7 in a human, the method comprising administering to a human in need thereof a pharmaceutically effective amount of an anti-HER-1 antibody.

Further provided is a method of treatment for triple negative breast cancer (TNBC) with GRB7 in a human, the method comprising administering to a human in need thereof a pharmaceutically effective amount of an EGFR inhibitor.

Also provided is a method of inhibiting GRB7 activity in a human experiencing triple negative breast cancer, the method comprising administering to a human in need thereof a pharmaceutically effective amount of an anti-HER-1 antibody.

Also provided is a method of inhibiting GRB7 activity in a human experiencing triple negative breast cancer, the method comprising administering to a human in need thereof a pharmaceutically effective amount of an EGFR inhibitor.

Provided is also a method of treating HER-2 positive human breast cancer that is resistant to trastuzumab, the method comprising administering to a human in need thereof a pharmaceutically effective amount of an anti-HER-1 antibody.

Provided is also a method of treating HER-2 positive human breast cancer that is resistant to trastuzumab, the method comprising administering to a human in need thereof a pharmaceutically effective amount of an EGFR inhibitor.

Provided is also a method of treating HER-2 positive human breast cancer that is resistant to at least one inhibitor of the HER receptor family, the method comprising administering to a human in need thereof a pharmaceutically effective amount of an anti-HER-1 antibody.

Provided is also a method of treating HER-2 positive human breast cancer that is resistant to at least one inhibitor of the HER receptor family, the method comprising administering to a human in need thereof a pharmaceutically effective amount of an EGFR inhibitor.

Provided is also a method of treating HER-2 positive human breast cancer that is resistant to at least one inhibitor of the HER receptor family, including those selected from the group of lapatinib, neratinib, and afatinib, the method comprising administering to a human in need thereof a pharmaceutically effective amount of an anti-HER-1 antibody.

Provided is also a method of treating HER-2 positive human breast cancer that is resistant to at least one inhibitor of the HER receptor family, including those selected from the group of lapatinib, neratinib, and afatinib, the method comprising administering to a human in need thereof a pharmaceutically effective amount of an EGFR inhibitor.

Provided is also a method of treating HER-2 positive human breast cancer that is resistant to both trastuzumab and at least one inhibitor of the HER receptor family, the method comprising administering to a human in need thereof a pharmaceutically effective amount of an anti-HER-1 antibody.

Provided is also a method of treating HER-2 positive human breast cancer that is resistant to both trastuzumab and at least one inhibitor of the HER receptor family, the method comprising administering to a human in need thereof a pharmaceutically effective amount of an EGFR inhibitor.

Provided is also a method of treating HER-2 positive human breast cancer that is resistant to both trastuzumab and at least one inhibitor of the HER receptor family, including those selected from the group of lapatinib, neratinib, and afatinib, the method comprising administering to a human in need thereof a pharmaceutically effective amount of an anti-HER-1 antibody.

Provided is also a method of treating HER-2 positive human breast cancer that is resistant to both trastuzumab and at least one inhibitor of the HER receptor family, including those selected from the group of lapatinib, neratinib, and afatinib, the method comprising administering to a human in need thereof a pharmaceutically effective amount of an EGFR inhibitor.

Also provided is a method of treating HER-2 positive breast cancer in a human, the method comprising administering to a human in need thereof:
a) a pharmaceutically effective amount of panitumumab;
b) a pharmaceutically effective amount of trastuzumab; and
c) a pharmaceutically effective amount of at least one inhibitor of the HER receptor family, including those selected from the group of lapatinib, neratinib, and afatinib, tor a pharmaceutically acceptable salt thereof.

Also provided is a method of treating HER-2 positive breast cancer in a human, the method comprising administering to a human in need thereof:
d) a pharmaceutically effective amount of an EGFR inhibitor;
e) a pharmaceutically effective amount of trastuzumab; and
f) a pharmaceutically effective amount of at least one inhibitor of the HER receptor family, including those selected from the group of lapatinib, neratinib, and afatinib, tor a pharmaceutically acceptable salt thereof.

Also provided is a method of treating HER-2 positive breast cancer in a human, the method comprising administering to a human in need thereof:
g) a pharmaceutically effective amount of panitumumab;
h) a pharmaceutically effective amount of trastuzumab; and
i) a pharmaceutically effective amount of lapatinib, or a pharmaceutically acceptable salt thereof.

Also provided is a method of treating HER-2 positive breast cancer in a human, the method comprising administering to a human in need thereof:
j) a pharmaceutically effective amount of an EGFR inhibitor;
k) a pharmaceutically effective amount of trastuzumab; and
l) a pharmaceutically effective amount of lapatinib, or a pharmaceutically acceptable salt thereof.

Provided herein is a method of treatment for therapy-resistant HER-2 positive breast cancer in a human, the method comprising administering to a human in need thereof
a) a pharmaceutically effective amount of an anti-HER-1 antibody;
b) a pharmaceutically effective amount of a second agent selected from an anti-HER-2 antibody and a toxin-conjugated anti-HER-2 antibody; and
c) a pharmaceutically effective amount of an anti-HER tyrosine kinase inhibitor.

Provided herein is a method of treatment for therapy-resistant HER-2 positive breast cancer in a human, the method comprising administering to a human in need thereof
a) a pharmaceutically effective amount of panitumumab;
b) a pharmaceutically effective amount of trastruzumab; and
c) a pharmaceutically effective amount of pertuzumab.

Provided herein is a method of treatment for therapy-resistant HER-2 positive breast cancer in a human, the method comprising administering to a human in need thereof
a) a pharmaceutically effective amount of an EGFR inhibitor;
b) a pharmaceutically effective amount of trastruzumab; and
c) a pharmaceutically effective amount of pertuzumab.

Provided herein is a method of treatment for therapy-resistant HER-2 positive breast cancer in a human, the method comprising administering to a human in need thereof
a) a pharmaceutically effective amount of panitumumab; and
b) a pharmaceutically effective amount of trastuzumab emtansine (TDM1).

Provided herein is a method of treatment for therapy-resistant HER-2 positive breast cancer in a human, the method comprising administering to a human in need thereof
a) a pharmaceutically effective amount of an EGFR inhibitor; and
b) a pharmaceutically effective amount of trastuzumab emtansine (TDM1).

Provided herein is a method of treatment for therapy-resistant HER-2 positive breast cancer in a human, wherein the therapy-resistant HER-2 positive breast cancer is metastatic, method comprising administering to a human in need thereof:
a) a pharmaceutically effective amount of panitumumab; and
b) a pharmaceutically effective amount of trastuzumab emtansine (TDM1).

Provided herein is a method of treatment for therapy-resistant HER-2 positive breast cancer in a human, wherein the therapy-resistant HER-2 positive breast cancer is metastatic, method comprising administering to a human in need thereof:
a) a pharmaceutically effective amount of an EGFR inhibitor; and
b) a pharmaceutically effective amount of trastuzumab emtansine (TDM1).

Provided herein is a method of treatment for therapy-resistant HER-2 positive breast cancer in a human, the method comprising administering to a human in need thereof
c) a pharmaceutically effective amount of panitumumab;
d) a pharmaceutically effective amount of trastuzumab emtansine (TDM1); and
e) a pharmaceutically effective amount of pertuzumab.

Provided herein is a method of treatment for therapy-resistant HER-2 positive breast cancer in a human, the method comprising administering to a human in need thereof
a) a pharmaceutically effective amount of an EGFR inhibitor;
b) a pharmaceutically effective amount of trastuzumab emtansine (TDM1); and
c) a pharmaceutically effective amount of pertuzumab.

Provided herein is a method of treatment for therapy-resistant HER-2 positive breast cancer in a human, wherein the therapy-resistant HER-2 positive breast cancer is metastatic, method comprising administering to a human in need thereof:
c) a pharmaceutically effective amount of panitumumab;
d) a pharmaceutically effective amount of trastuzumab emtansine (TDM1); and
e) a pharmaceutically effective amount of pertuzumab.

Provided herein is a method of treatment for therapy-resistant HER-2 positive breast cancer in a human, wherein the therapy-resistant HER-2 positive breast cancer is metastatic, method comprising administering to a human in need thereof:
a) a pharmaceutically effective amount of an EGFR inhibitor;
b) a pharmaceutically effective amount of trastuzumab emtansine (TDM1); and
c) a pharmaceutically effective amount of pertuzumab.

Provided herein is a method of treatment for therapy-resistant HER-2 positive breast cancer in a human, the method comprising administering to a human in need thereof:
d) a pharmaceutically effective amount of panitumumab;
e) a pharmaceutically effective amount of trastruzumab;
f) a pharmaceutically effective amount of pertuzumab; and
g) a pharmaceutically effective amount of an anti-HER tyrosine kinase inhibitor.

Provided herein is a method of treatment for therapy-resistant HER-2 positive breast cancer in a human, the method comprising administering to a human in need thereof:
a) a pharmaceutically effective amount of an EGFR inhibitor;
b) a pharmaceutically effective amount of trastruzumab;
c) a pharmaceutically effective amount of pertuzumab; and

a pharmaceutically effective amount of an anti-HER tyrosine kinase inhibitor

Provided herein is a method of treatment for therapy-resistant HER-2 positive breast cancer in a human, wherein the therapy-resistant HER-2 positive breast cancer is metastatic, method comprising administering to a human in need thereof:
a) a pharmaceutically effective amount of panitumumab;
b) a pharmaceutically effective amount of trastruzumab;
c) a pharmaceutically effective amount of pertuzumab; and
d) a pharmaceutically effective amount of an anti-HER tyrosine kinase inhibitor.

Provided herein is a method of treatment for therapy-resistant HER-2 positive breast cancer in a human, wherein the therapy-resistant HER-2 positive breast cancer is metastatic, method comprising administering to a human in need thereof:
a) a pharmaceutically effective amount of an EGFR inhibitor;
b) a pharmaceutically effective amount of trastruzumab;
c) a pharmaceutically effective amount of pertuzumab; and
d) a pharmaceutically effective amount of an anti-HER tyrosine kinase inhibitor.

In one embodiment of the methods herein, the anti-HER tyrosine kinase inhibitor is lapatinib. In other embodiments the anti-HER tyrosine kinase inhibitor is selected from the group of lapatinib, neratinib, afatinib, and gefitinib.

Provided herein is another method of treatment for therapy-resistant HER-2 positive breast cancer in a human, the method comprising administering to a human in need thereof
a) a pharmaceutically effective amount of an anti-HER-1 antibody;
b) a pharmaceutically effective amount of a second agent selected from an anti-HER-2 antibody and a toxin-conjugated anti-HER-2 antibody;
c) a pharmaceutically effective amount of an anti-HER tyrosine kinase inhibitor; and
d) a pharmaceutically effective amount of an anti-HER-3 antibody.

Provided herein is another method of treatment for therapy-resistant HER-2 positive breast cancer in a human, the method comprising administering to a human in need thereof
e) a pharmaceutically effective amount of an EGFR inhibitor;
f) a pharmaceutically effective amount of a second agent selected from an anti-HER-2 antibody and a toxin-conjugated anti-HER-2 antibody;
g) a pharmaceutically effective amount of an anti-HER tyrosine kinase inhibitor; and
h) a pharmaceutically effective amount of an anti-HER-3 antibody.

Also provided is a method of reducing therapy resistance in therapy-resistant breast cancer cells in a human in need thereof, the method comprising administering to the human a pharmaceutically effective amount of an anti-HER-1 antibody.

Also provided is a method of reducing therapy resistance in therapy-resistant breast cancer cells in a human in need thereof, the method comprising administering to the human a pharmaceutically effective amount of panitumumab.

Examples of EGFR inhibitors that may be used with the methods herein include erlotinib)(TARCEVA®), gefitinib (IRESSA®), Lapatinib (TYKERB®), panitumumab) (VECTIBIX®), vandetanib (CAPRELSA®), neratinib (NERLYNX®), necitumumab) (PORTRAZZA®), and osimertinib (TAGRISSO®). Within each of the methods herein using an EGFR inhibitor, there is a further embodiment in which the EGFR inhibitor is erlotinib, or a pharmaceutically acceptable salt thereof.

Erlotinib may be administered in the methods herein at a dose of from about 25 mg/day to about 200 mg/day. In separate independent embodiments, erlotinib may be administered to a subject in need thereof in the present methods at doses selected from the group of about 25 mg/day, about 50 mg/day, about 75 mg/day, about 100 mg/day, about 125 mg/day, about 150 mg/day, about 175 mg/day, and about 200 mg/day.

Within each of the methods herein using an EGFR inhibitor, there is a further embodiment in which the EGFR inhibitor is gefitinib, or a pharmaceutically acceptable salt thereof.

Within each of the methods herein using an EGFR inhibitor, there is a further embodiment in which the EGFR inhibitor is lapatinib, or a pharmaceutically acceptable salt thereof.

Within each of the methods herein using an EGFR inhibitor, there is a further embodiment in which the EGFR inhibitor is panitumumab, or a pharmaceutically acceptable salt thereof.

Within each of the methods herein using an EGFR inhibitor, there is a further embodiment in which the EGFR inhibitor is vandetanib, or a pharmaceutically acceptable salt thereof.

Within each of the methods herein using an EGFR inhibitor, there is a further embodiment in which the EGFR inhibitor is neratinib, or a pharmaceutically acceptable salt thereof. In some embodiments, neratinib is administered to adult human subjects at a dose of from about 50mg/day to about 400 mg/day. In other embodiments, it is administered at a daily dose of from about 75 mg to about 300 mg. In some additional embodiments, neratinib is administered at a daily dose of from about 200 mg to about 300 mg.

Within the methods herein, erlotinib may be administered at a daily dose of from about 25 mg to about 200 mg. In separate individual embodiments, erlotinib may be administered in daily doses selected from the group of about 25 mg, about 50 mg, about 75 mg, about 100 mg, about 125 mg, about 150 mg, about 175 mg, and about 200 mg.

Within the methods herein, gefitinib may be administered at a daily dose of from about 50 mg to about 400 mg. In other embodiments, gefitinib may be administered at from about 150 mg/day to about 350 mg/day. In additional embodiments, gefitinib is administered at a daily dose of from about 200 mg to about 300 mg. In separate individual embodiments, gefitinib may be administered in daily doses selected from the group of about about 50 mg, about 75 mg, about 100 mg, about 125 mg, about 150 mg, about 175 mg, about 200 mg, about 250 mg, about 300 mg, about 350 mg, and about 400 mg.

Lapatinib may be administered to a subject in the methods herein at a daily dose of from about 500 mg to about 2,000 mg. In some embodiments, lapatinib may be administered at doses of from about 1,000 mg to about 1,500 mg daily. In some regimens, the daily dose of lapatinib is about 1,250 mg.

Vandetanib may be administered in the methods herein at doses of from about 50 mg/day to about 300 mg/day. In some embodiments, vandetanib is administered at a daily dose of from about 100 mg to about 300 mg.

Osimertinib may be administered in the methods herein at doses of from about 20 mg/day to about 100 mg/day. In some embodiments, osimertinib is administered at a dose of from about 40 mg/day to about 80 mg/day. In some embodiments, the daily dose is 40 mg. In other embodiments, the dose is 80 mg.

Afitinib may be administered in the methods herein at a daily dose of from about 20 mg to about 80 mg. In some embodiments, the daily dose of afitinib is from about 20 mg to about 40 mg.

Vemurafenib may be administered in the methods herein at a dose of about 200 mg to about 1,200 mg once or twice daily. In some embodiments, vemurafenib is administered at a dose of from about 800 mg to about 1,200 mg once or twice daily. In other embodiments, vemurafenib is administered at a dose of from about 900 mg to about 1,000 mg once or twice per day.

Cobimetinib may be administered in the methods herein at a daily dose of from about 20 mg/day to about 100 mg/day. In some embodiments, the daily dose is from about 40 mg/day to about 80 mg/day.

Canertinib (CI-1033) may be administered in methods herein at a daily dose of from about 20 mg to about 50 mg.

Dacomitinib (marketed as VIZIMPRO® tablets) may be administered in the methods herein at a daily dose of from about 15 mg to about 60 mg. In some embodiments, dacomitinib is administered at a daily dose of about 45 mg.

Patritumab may be administered in the methods herein at a dose of from about 5 mg/day to about 50 mg/day. In some embodiments, patritumab is administered at a daily dose of from about 5 mg/day to about 25 mg/day.

Seribantumab (MM-121) may be administered in the methods herein at an initial dose of from about 10 mg/kg to about 60 mg/kg, followed by weekly doses of from about 10 mg/kg to about 40 mg/kg. In some embodiments, an initial seribantumab dose of from about 25 mg to about 50 mg is followed by weekly doses of from about 10 mg/kg to about 30 mg/kg.

Within each of the methods herein using an EGFR inhibitor, there is a further embodiment in which the EGFR inhibitor is necitumumab, or a pharmaceutically acceptable salt thereof.

Within each of the methods herein using an EGFR inhibitor, there is a further embodiment in which the EGFR inhibitor is osimertinib, or a pharmaceutically acceptable salt thereof.

Examples of anti-HER-2 antibodies useful in the methods herein include, but are not limited to, trastuzumab (HERCEPTIN®) and pertuzumab (PERJETA®). Examples of anti-HER-3 antibodies useful in the methods herein include patritumab (AMG-888), GSK2849330 (GlaxoSmithKline), seribantumab (MM-121), lumertuzumab (RG7116, Roche), UM716 (Novartis), AV-203 (Aveo Oncology), REGN1400 (Regeneron), MM-111 (Merrimack), MM-141 (Merrimack), and duligotuzumab (RG7597, Roche).

Examples of toxin-conjugated anti-HER-2 antibodies that may be used in the methods described herein includes, but are not limited to, trastuzumab-emtansine (ado-trastuzumabe emtansine or T-DM1).

Examples of anti-HER tyrosine kinase inhibitors that may be used in the present methods include, but are not limited to, lapatinib, neratinib, afatinib, gefitinib, erlotinib, vemurafinib, cobimetinib, AZD8931, canertinib (CI-1033), CP-724714, CUDC-101, dacomitinib (PF299804), pelitinib (EKB-569), AST-1306, TAK-285, AC-480 (BMS-599626) and compounds of the structures:

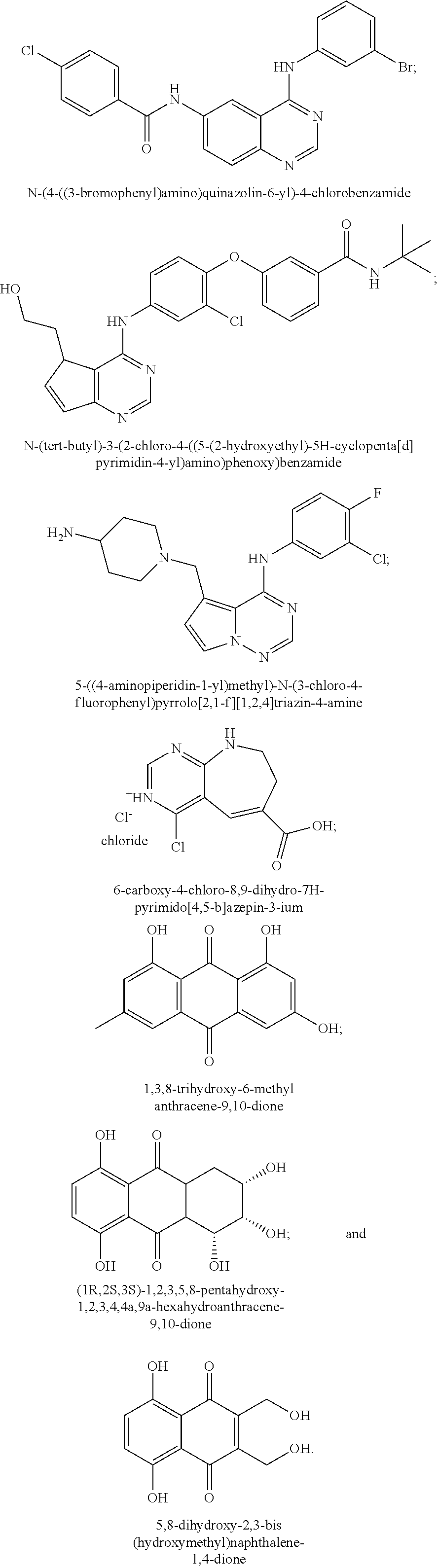

In some embodiments of the methods herein, the breast cancer is locally advanced TNBC, such as, for example, stage III TNBC (including stages IIIA, IIIB and IIIC TNBC). In another embodiment, breast cancer is metastatic TNBC (i.e. stage IV TNBC). In one embodiment, TNBC is inflammatory breast cancer.

In other embodiments, breast cancer is inflammatory breast cancer (IBC).

In other embodiments, breast cancer is advanced IBC. In one embodiment, breast cancer is locally advanced IBC. In another embodiment, breast cancer is metastatic IBC.

In other embodiments, inflammatory breast cancer is triple-negative inflammatory breast cancer.

In some embodiments, the human patient was not treated previously with another treatment for breast cancer (i.e. one of the methods herein is the first line treatment). In one embodiment, the subject was not treated previously with another systemic treatment for breast cancer (wherein a systemic treatment for breast cancer relates to treatment with a chemotherapeutic agent). In one embodiment, the subject was not previously treated for breast cancer by surgery or radiotherapy.

In other embodiments, the subject previously received one, two or more other treatments for breast cancer (i.e. the method of the invention is a second line, a third line or more). In one embodiment, the subject previously received one or more other treatments for breast cancer, but was unresponsive or did not respond adequately to these treatments, which means that there is no or too low therapeutic benefit induced by these treatments. Therapeutic benefits may include the fact of (1) slowing down or stopping the progression, aggravation, or deterioration of one or more symptoms of breast cancer; (2) bringing about ameliorations of the symptoms of breast cancer; (3) reducing the severity or incidence of breast cancer; or (4) curing breast cancer,

Treatments for breast cancer include, but are not limited to, surgery (lumpectomy, partial or total mastectomy, modified radical mastectomy, lymph node surgery), tumor ablation, radiation therapy, chemotherapy (including treatment with the agents listed below), hormone therapy (including treatment with tamoxifen, estrogens or aromatase inhibitors), targeted therapy (including treatment with monoclonal antibodies (such as, for example, trastuzumab (especially in HER2 plus breast cancer), pertuzumab or ado-trastuzumab emtansine), other tyrosine kinase inhibitors than masitinib (such as, for example, lapatinib), and PARP inhibitors).

Examples of chemotherapeutic agents that may be used for treating breast cancer in combination with an anti-HER-1 antibody, such as panitumumab, include, but are not limited to, abemaciclib, ahitrexate (Methotrexate), abraxane (Paclitaxel Albumin-stabilized Nanoparticle Formulation), ado-Trastuzumab Emtansine, adrucil (Fluorouracil), afinitor (Everolimus), anastrozole, aredia (Pamidronate Disodiurn), arimidex (Anastrozole), aromasin (Exemestane), carboplatin, capecitabine, cisplatin, Clafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride (Ellence), Eribulin Mesylate, Everolimus, Exemestane, Fareston (Toremifene), Faslodex (Fulvestrant), Femara (Letrozole), Fluoroplex (Fluorouracil), Fluorouracil, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ixabepilone, lxempra (Ixabepilone), Kadcyla (Ado-Trastuzurnab Erntansine), Lapatinib Ditosylate, Letrozole, Megace (Megestrol Acetate), Megestrol Acetate, Methotrexate, Methotrexate LPF (Methotrexate), Mexate (Methotrexate), Mexate-AQ (Methotrexate), mitoxantrone, Neosar (Cyclophosphamide), Neratinib Maleate (Nerlynx), Nolvadex (Tamoxifen Citrate), Paclitaxel, Paclitaxel Albumin-stabilized Nanoparticle Formulation, palbociclib (lbrance), Pamidronate Disodiurn, Perjeta (Pertuzumah), Pertuzumab, ribociclib, Tamoxifen Citrate, Taxol (Paclitaxel), Taxotere (Docetaxel), Trastuzurnab, Toremifene, Tykerb (Lapatinib Ditosylate), Velban (Vinblastine Sulfate), Velsar (Vinblastine Sulfate), Vinblastine Sulfate, Xeloda (Capecitabine), Zoladex (Goserelin Acetate), Irinotecan, platinum-based chemotherapy, antimetabolites, anthracyclines and taxanes. Useful in the methods herein are the combinations doxorubicin HCl (Adriamycin)/cyclophosphamide (AC); doxorubicin HCl/cyclophosphamide/paclitaxal (Taxol) (ACT); cyclophosphamide/doxorubicin HCl/fluoruracil (CAF); cyclophosphamide/methotrexate/fluroruacil (CMF); fluorocuracil/epirubicin HCl/cyclophosphamide (FEC); and docetaxel (Taxotere)/Doxorubicin HCl (Adriamycin)/cyclophosphamide (TAC).

### Definitions

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

As used herein, the term “breast cancer” refers to all subtypes of invasive breast carcinomas, including triple negative breast cancer and inflammatory breast cancer.

The terms “inhibiting” or “inhibition” indicates a decrease, such as a significant decrease, in the baseline activity of a biological activity or process. For example, “inhibition of HER-1 activity” refers to a decrease in HER-1 signaling, expression, or activity as a direct or indirect response to the presence of a compound or agent, relative to the activity of HER-1 in the absence of such compound or a pharmaceutically acceptable salt or co-crystal thereof. The decrease in activity may be due to the direct interaction of the compound with HER-1, or due to the interaction of the compound(s) described herein with one or more other factors that in turn affect HER-1 activity. For example, the presence of the compound(s) or agent(s) may decrease HER-1 activity by directly binding to the HER-1, by causing (directly or indirectly) another factor to decrease HER-1 activity, or by (directly or indirectly) decreasing the amount of HER-1 present in the cell or organism. In some embodiments, the inhibition of HER-1 activity may be compared in the same subject prior to treatment, or other subjects not receiving the treatment.

“Triple-negative breast cancer” (TNBC) is characterized by the absence of the estrogen-receptor (ER) and of the progesterone-receptor (PR), and by the absence of overexpression of the human epidermal growth factor receptor type 2 (HER2). TNBC has been associated with high GRB7 RNA expression levels. In each embodiment herein for the treatment of TNBC, there is a further embodiment comprising the same steps of the method in which the TNBC is associated with high GRB7 RNA expression levels. High levels refers to higher than median levels determined in GRB7-overexpressing individuals as determined by immunohistochemistry or reverse transcription polymerase chain reaction (RTPCR). In some embodiments high levels of GRB7 expression concerns the upper one third of a population of GRB7-overexpressing individuals.

The terms “knock down”, “knockdown”, and “knocking down” refer to an experimental technique by which expression of one or more of an organisms genes are reduced.

The term “therapeutically effective amount” or “pharmaceutically effective amount” refers to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of panitumumab is an amount sufficient to modulate HER-1 signaling, expression or activity, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of HER-1 signaling activity.

“Treatment” or “treating” is an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

“Delaying” refers to the development of a disease or condition means to defer, hinder, slow, retard, stabilize, and/or postpone development of the disease or condition. This delay can be of varying lengths of time, depending on the history of the disease or condition, and/or subject being treated. A method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

By “significant” is meant any detectable change that is statistically significant in a standard parametric test of statistical significance such as Student's T-test, where p<0.05.

“Pharmaceutically acceptable salts”, such as those referenced for lapatinib, include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

“Subject” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula I, or a pharmaceutically acceptable salt or co-crystal thereof, as described herein. This includes a subject who may be determined to be at risk of or susceptible to such diseases or conditions, such that treatment would prevent the disease or condition from developing.

Panitumumab, formerly known as ABX-EGF, is a fully humanized monoclonal antibody against HER-1 (EGFR) approved for the treatment of colon cancer and available under the tradename Vectibix® solution for intravenous infusion. For each of the methods described herein there are further embodiments in which the anti-HER-1 antibody is panitumumab. Lapatinib is available as lapatinib ditosylate under the tradenames Tykerb® and Tyverb®

While the appropriate dose and dosing schedule for each drug agent used in the combinations and methods herein will be determined by an appropriate medical professional, panitumumab may be administered in some regimens of the methods herein at from about 2 mg/kg to about 10 mg/kg every 14 days as an intravenous infusion. In some embodiments, panitumumab may be administered in some regimens at from about 4 mg/kg to about 8 mg/kg every 14 days as an intravenous infusion. In other embodiments, panitumumab may be administered in some regimens at from about 5 mg/kg to about 7 mg/kg every 14 days as an intravenous infusion. In further embodiments, panitumumab may be administered in some regimens at about 6 mg/kg every 14 days as an intravenous infusion.

Trastuzumab may be administered in some regimens in the methods herein at from about 2 mg/kg to about 15 mg/kg from once every week to once every three weeks as an intravenous infusion. In some embodiments, trastuzumab may be administered as an initial 90 minute IV infusion at an initial dose of from about 2mg/kg to about 6 mg/kg, followed by a second IV infusion over about 30 minutes at a dose of from about 1 mg/kg to about 3 mg. In further embodiments, an initial dose of 4 mg/kg is administered to the subject by IV infusion over about 90 minutes, followed by a 30 minute IV infusion of about 2 mg/kg.

Pertuzumab may be administered in some regimens in the methods herein at an initial 60-minute IV infusion of from about 600 mg to about 1,000 mg, followed in 3-week increments thereafter as an IV infusion of from about 300 mg to about 600 mg. In some embodiments, the initial 60-minute pertuzumab IV infusion of from about 750 mg to about 900 mg, followed once every three weeks thereafter by a pertuzumab IV infusion of from about 350 mg to about 500 mg. In other embodiments, an initial 60-minute pertuzumab IV infusion of 840 mg is given, followed in 3-week increments by subsequent 30-60-minute infusions of about 420 mg of pertuzumab.

In some embodiments, an initial 90 minute IV infusion of from about 6 mg/kg to about 10 mg/kg of trastuzumab is given, followed by a 30-90 minute IV trastuzumab infusion at a dose of from about 4 mg/kg to about 8 mg/kg. In other embodiments trastuzumab is administered in a two-stage IV infusion, with about 8 mg/kg being administered in an initial 90 minute session, followed by about 6 mg/kg of trastuzumab over 30-90 minutes of IV infusion.

Lapatinib may be administered at a dosage of from about 500 mg/day to about 1,500 mg/day. In some embodiments the lapatinib is administered for 21 consecutive days.

### Materials and Methods

The GRB7 gene encodes a multi-domain signal transduction molecule that is part of the core of the HER-2 amplicon. GRB7 is commonly co-amplified and overexpressed with HER-2 in human breast cancers. Though previous studies have found a functional role for GRB7 in breast cancer, the role of GRB7 in human HER-2 positive targeted-therapy resistant disease remains unexplored. In his study HER-2 positive, trastuzaumab and lapatinib treatment resistant cell lines were utilized to explore the role of GRB7 signaling in cell proliferation in cell culture and tumor xenographs. We found that GRB7 signaling remains important to cell proliferation even as HER-2 positive cancer cells develop resistant to HER-2 targeted therapy, and that GRB7 signaling is mediated, at least in part by HER-1

HCC-1954, 21MT1 and JimT1 are human HER-2 positive breast cancer cell lines that are resistant to trastuzumab and lapatinib treatment. Transient knock down of GRB7 protein expression was achieved with siRNA transfection and stable knock down with lentiviral vector mediated shRNA over-expression. Cell lines transfected with non-targeting siRNA or shRNA serve as negative controls. GRB7 knock down is verified by Western blotting. The growth of human breast cancer cell lines after GRB7 knock down in vitro is measured with the CellTiter Glo assay as well as the Incucyte live cell imaging. Activation status of specific signaling pathways was examined with phospho-specific antibody by immune-blotting and immune-precipitation. To assess the growth promoting function of GRB7 in human breast cancer cell lines in vivo, polyclonal HCC-1954, 21MT1 and JimT1 cells, with GRB7 knock down or their corresponding negative control, were orthotopically injected into the mammary fat pads of female immune-deficient NSG mice. The growth rates of these tumors, measured serially with caliper, and final tumor weights were compared between GRB7 knock down and the negative control. The proliferation rate and apoptosis of these tumors were studied with ki-67 staining and Tunel assay. The effects of GRB7 knock down on signaling were investigated with a proteome profiler receptor tyrosine kinase kit (R&D). The role of HER-1 signaling in the growth of parental HCC1954, 21MT1 and JimT1 cells are examined by transient knock down of HER-1 expression with siRNA transfection and treatment with a humanized anti-antibody, panitumumab.

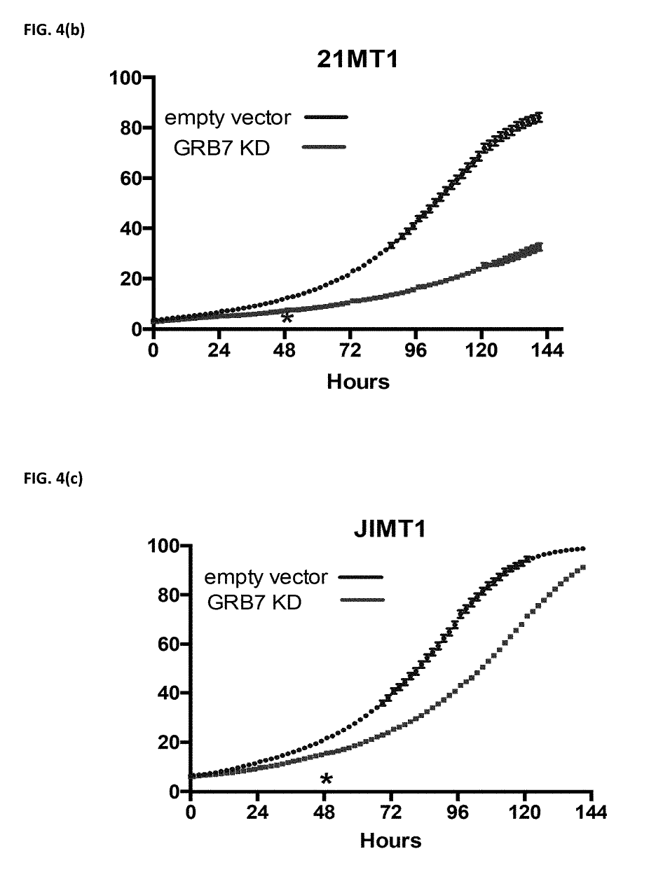

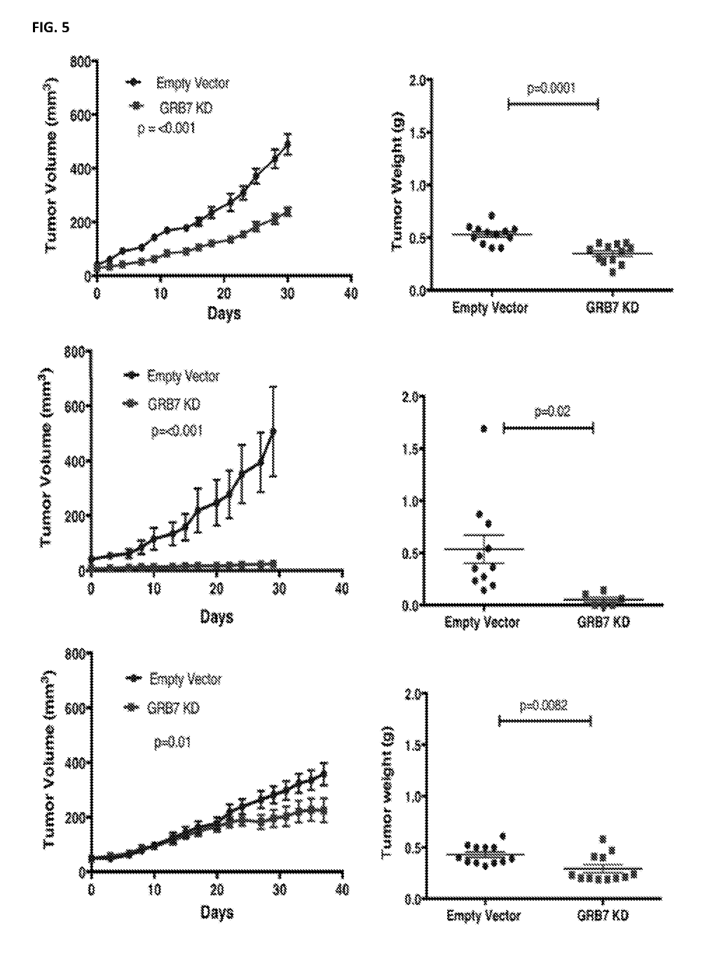

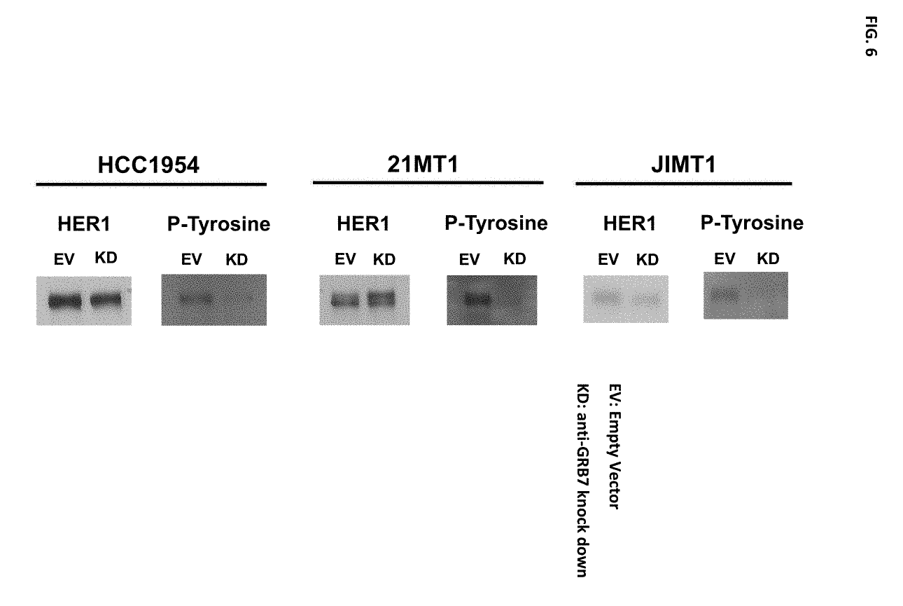

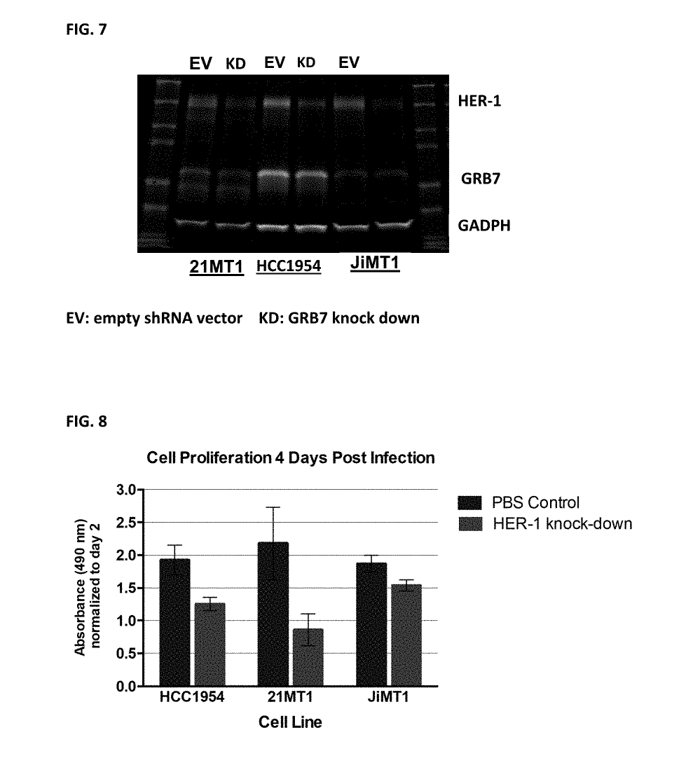

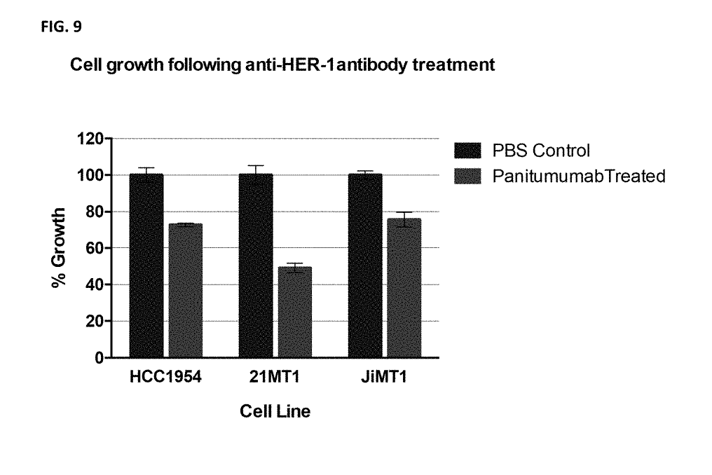

Results- GRB7 knock down decreases the growth of HCC-1954, 21MT1 and JimT1 cells in vitro. GRB7 knock down also reduces the growth of tumor xenograft in animal models. When assayed by ki67 staining and Tunel assay, the mechanism of reduced tumor xenograft growth appears to be distinct among all three cellular contexts. Reduced proliferation and increased apoptosis were seen in 21MT1 cells, while only reduced proliferation was seen in HCC-1954 cells and only increased apoptosis was seen in JimT1 cells, respectively. Profiling found HER-1 tyrosine phosphorylation was down with GRB7 knock down in JimT1 cells. Immuno-blotting and immuno-precipitation experiments found HER-1 phosphorylation was reduced with GRB7 knock down in all three cell lines, as indicated in FIGS. 4(a), 4(b), and 4(c). In each graph, the empty vector is plotted in the upper line and the GRB7 knock down is represented by the lower. GRB7 knock down via siRNA transient transfection as well as blocking HER-1 function with panitumumab decreased proliferation of all three parental cell lines in vitro.

Discussion- GRB7 has essential growth promoting function in therapy resistant HER-2 positive human breast cancer cells. GRB7 knock down has pleiotropic effects on signaling in various cellular contexts. The growth promoting effect of GRB7 is mediated in part by HER-1 activation; blocking HER-1 function therefore represents a novel therapeutic strategy in the treatment of therapy resistant HER-2 positive breast cancer.

What is claimed:

1. A method of treatment for therapy-resistant HER-2 positive breast cancer in a human, the method comprising administering to a human in need thereof a pharmaceutically effective amount of an anti-HER-1 antibody.

2. The method of claim 1 comprising further reducing GRB7 activity in the human experiencing a therapy-resistant HER-2 positive breast cancer in a human.

3. The method of claim 1 wherein the HER-2 positive human breast cancer is resistant to trastuzumab.

4. The method of claim 1 wherein the HER-2 positive human breast cancer is resistant to at least one inhibitor of the HER receptor family from the group of lapatinib, neratinib, and afatinib.

5. The method of claim 1 wherein the HER-2 positive human breast cancer is resistant to both trastuzumab and at least one inhibitor of the HER receptor family selected from the group of lapatinib, neratinib, and afatinib.

6. A method of treating HER-2 positive breast cancer in a human, the method comprising administering to a human in need thereof:
g) a pharmaceutically effective amount of panitumumab;
h) a pharmaceutically effective amount of trastuzumab; and
i) a pharmaceutically effective amount of at least one inhibitor of the HER receptor family selected from the group of lapatinib, neratinib, and afatinib, tor a pharmaceutically acceptable salt thereof.

7. The method of claim 6 wherein the HER-2 positive human breast cancer is resistant to at least one inhibitor of the HER receptor family.

8. The method of claim 6 wherein the HER-2 positive human breast cancer is resistant to at least one inhibitor of the HER receptor family selected from the group of lapatinib, neratinib, and afatinib, or a pharmaceutically acceptable salt thereof.

9. The method of claim 6 wherein the HER-2 positive human breast cancer is resistant to both trastuzumab and at least one inhibitor of the HER receptor family selected from the group of lapatinib, neratinib, and afatinib, or a pharmaceutically acceptable salt thereof.

10. A method of treatment for therapy-resistant HER-2 positive breast cancer in a human, the method comprising administering to a human in need thereof
a) a pharmaceutically effective amount of an anti-HER-1 antibody;
b) a pharmaceutically effective amount of a second agent selected from an anti-HER-2 antibody and a toxin-conjugated anti-HER-2 antibody; and
c) a pharmaceutically effective amount of an anti-HER tyrosine kinase inhibitor.

11. The method of claim 10 comprising administering to the human in need thereof
a) a pharmaceutically effective amount of panitumumab;
b) a pharmaceutically effective amount of trastruzumab; and
c) a pharmaceutically effective amount of pertuzumab.

12. The method of claim 10, wherein the anti-HER-1 antibody is panitumumab.

13. The method of claim 6, wherein the inhibitor of the HER receptor family is lapatinib, or a pharmaceutically acceptable salt thereof.

14. The method of claim 10, wherein the anti-HER tyrosine kinase inhibitor is selected from the group of lapatinib, neratinib, afatinib, and gefitinib, or a pharmaceutically acceptable salt thereof.

15. The method of claim 10, wherein the anti-HER tyrosine kinase inhibitor is selected from the group of lapatinib, neratinib, afatinib, gefitinib, erlotinib, vemurafinib, cobimetinib, AZD8931, canertinib (Cl-1033), CP-724714, CUDC-101, dacomitinib (PF299804), perlitinib (EKB-569, AST-1306, TAK-285, AC-480 (BMS-599626) and compounds of the structures: 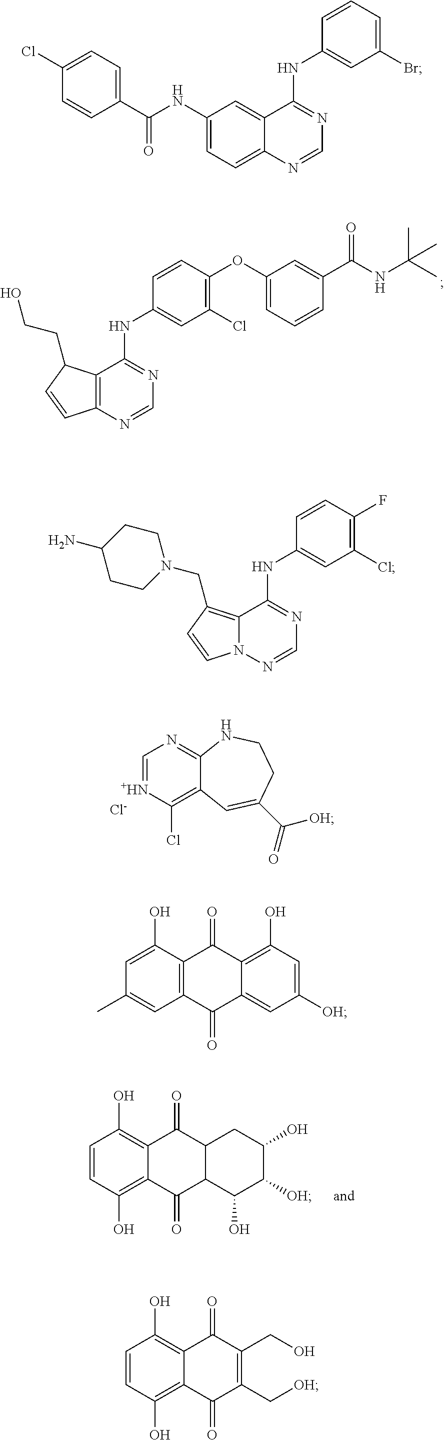
or a pharmaceutically acceptable salt thereof.

16. The method of claim 14, wherein the anti-HER tyrosine kinase inhibitor is lapatinib, or a pharmaceutically acceptable salt thereof.

17. The method claim 12 the panitumumab is administered to the human in need thereof at from dose of from about 2 mg/kg to about 10 mg/kg.

18. The method of claim 13, wherein the lapatinib, or a pharmaceutically acceptable salt thereof, is administered to the human in need thereof at from dose of from about 500 mg/day to about 1,500 mg/day
